(19) United States
(12) Patent Application Publication
(10) Pub. No.: US 2002/0188182 A1
(43) Pub. Date: Dec. 12, 2002

(54) System and method for scoring and managing patient progression

(76) Inventors: John Edward Haines, Lantana, FL (US); Linda L. Roman, Boca Raton, FL (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 09/878,622

(22) Filed: Jun. 11, 2001

(51) Int. Cl. A61B 5/00

(52) U.S. Cl. 600/300

(57) ABSTRACT

A system and method for scoring and managing patient progression is disclosed. A transceiver provides communication capabilities between a patient, a care provider, and/or a physician. A memory is communicably coupled to the transceiver, having logic therein that defines functions to be performed by the system. A processor is configured by the memory to perform the functions of: prescribing a care plan for the patient based upon medical conditions of the patient; performing an initial medical assessment of the patient; performing a medication compliance assessment of the patient; modifying the care plan based upon the initial assessment and the medication compliance assessment; and modifying the care plan in accordance with new assessments performed in association with medical progress of the patient. Answers to questions asked during the initial medical assessment of the patient, and during the new assessments, are scored for comparison purposes, thereby providing an accurate mechanism for monitoring patient progression.

### FIELD OF THE INVENTION

The present invention generally relates to medical patient therapy and, more particularly, is related to a system and method for tracking and scoring patient progression toward a desired medical outcome via use of a networked environment.

### BACKGROUND OF THE INVENTION

Patient care typically has one of three goals, the return to health of a patient, maintaining the current level of health of the patient, or, if the return to a truly healthy condition is not possible, to allow the patient to enjoy a high quality of life for their remaining years. Care provider activities are focused on helping the patient reach a positive outcome for their specific disease process. In many cases, there is not a smooth integration of multiple care providers or objective measurement of progress.

Traditional approaches to achieving a positive patient outcome are centered on medical treatment and care plans. Physicians typically adopt a treatment plan that is individualized based on the patient, the patient condition, the disease process, and the frame of reference of the physician. The resulting treatment plan becomes the basis for the care plan. While it may seem that the treatment plan is a formal process, the reality is that it is only a loosely defined and highly subjective document.

The care plan is the working plan for implementing the prescribed physician treatment plan and the application of accepted disease management protocols. The development and application of the care plan is highly individualized and outcomes are highly subjective. Since there is little business incentive to coordinate and integrate the efforts of a physician and a care provider, methodologies and actions used to provide patient care do not achieve their maximum efficiency. In some cases, the lack of coordination results in poorly set goals, medication conflicts, and sub-optimal patient recovery.

In order to increase the effectiveness of care plans in achieving measurable and more objective patient outcomes, care providers have begun to use stand-alone software tools. Case management and disease management tools allow for some measure of increased standardization of the development of a care plan. The standardization allows for a more objective assessment of the patient outcome by utilizing various methods, some including scoring criteria. However, the software tools are only utilized by the care provider and are based solely on objective and subjective observed data and retrospective input. All data used for scoring and assessment is developed on-site in contact with the patient or via telephonic interaction with the patient. The software tools are not integrated with the processes, methods, or systems of the physician or other care providers, and they are both after-the-fact and labor intensive.

Moreover, while these software tools have begun to address issues of standardization, they do not provide for integration of critical processes, standardized scored assessment methods, and/or necessary feedback loops. Management protocols are not tailored specifically to meet the individual needs of the patient. In many cases, the first line physician, principally the family practice physician, is only a casual observer to the care process and their activities are limited to office visits, review of the care plan, and periodic reports from the cost prohibitive efforts. Real time intervention and care adjustment is not possible without considerable and cost prohibitive efforts.

Thus, a heretofore-unaddressed need exists in the industry to address the aforementioned deficiencies and inadequacies.

### SUMMARY OF THE INVENTION

In light of the forgoing, the preferred embodiment of the present invention generally relates to a system and method for scoring and managing a medical patient's progression in order to decrease patient recovery time.

Generally, describing he structure of the system, the system utilizes a transceiver for providing communication capabilities between a patient, a care provider, and/or a physician. A memory is communicably coupled to the transceiver, having logic therein that defines functions to be performed by the system. A processor is configured by the memory to perform the functions of prescribing a care plan for the patient that is predefined and based upon medical conditions of the patient; performing an initial medical assessment of the patient; performing a medication compliance assessment of the patient; modifying the care plan based upon the initial assessment and the medication compliance assessment; and modifying the care plan in accordance with new assessments performed in association with medical progress of the patient.

The present invention can also be viewed as providing a method for scoring and managing the progression of a medical patient in order to decrease patient recovery time. In this regard, the method can be broadly summarized by the following steps: determining whether the patient qualifies for participating in a program using the method; prescribing a care plan for the patient that is based upon medical conditions of the patient; performing an initial medical assessment of the patient; performing a medication compliance assessment of the patient; modifying the care plan based upon the initial assessment and the medication compliance assessment; and modifying the care plan in accordance with new assessments performed in association with medical progress of the patient.

The invention has numerous advantages, a few of which are delineated hereafter as examples. Note that the embodiments of the invention, which are described herein, possess one or more, but not necessarily all, of the advantages set out hereafter. One advantage of the invention is that it provides a mechanism for accurately managing patient medical progression, thereby assisting in patient recovery.

Another advantage is that the invention provides for managing of patient medical progression without the requiring that the patient leave the presence of their home.

Other features and advantages of the present invention will be or become apparent to one with skill in the art upon examination of the following drawings and detailed description. It is intended that all such additional features and advantages be included herein within the scope of the present invention, as defined by the accompanying claims.

### BRIEF DESCRIPTION OF THE DRAWINGS

The present invention will be more fully understood from the detailed description given below and from the accompanying drawings of the preferred embodiments of the invention, which, however, should not be taken to limit the invention to the specific embodiments, but are for explanation and better understanding. Furthermore, the drawings are not necessarily to scale, emphasis instead being placed upon clearly illustrating the principles of the present invention. Finally, like reference numerals in the figures designate corresponding parts throughout the several drawings.

FIG. 1 illustrates an Internet based system upon which the scoring and managing system of the present invention may be implemented.

FIG. 2 is a block diagram that further illustrates the patient unit of FIG. 1.

FIG. 3 is a block diagram that further illustrates the care provider digital processor of FIG. 1.

FIG. 4 is a block diagram that provides a logical depiction of the categories of data collected and maintained within the patient assessment process of the scoring and managing system of FIG. 1.

FIG. 5 is a flow chart that illustrates a logical path taken by the scoring and managing system of FIG. 1 to ensure rapid patient recovery.

### DETAILED DESCRIPTION OF THE PREFERRED EMBODIMENT

The system for scoring and managing patient progression can be implemented in software, firmware, hardware, or a combination thereof. In the preferred embodiment of the invention, which is intended to be a non-limiting example, a portion of the system is implemented in software that is executed by a computer, for example, but not limited to, a personal computer, workstation, mini computer, or mainframe computer.

The software-based portion of the system, which comprises an ordered listing of executable instructions for implementing logical functions, can be embodied in any computer-readable medium for use by, or in connection with, an instruction execution system, apparatus, or device such as a computer-based processor-containing system, or other system that can fetch the instructions from the instruction execution system, apparatus, or device and execute the instructions. In the context of this document, a “computer-readable medium” can be any means that can contain, store, communicate, propagate or transport the program for use by or in connection with the instruction execution system, apparatus or device. The computer-readable medium can be, for example, but not limited to, an electronic, magnetic, optical, electromagnetic, infrared, or semiconductor system, apparatus, device, or propagation medium. More specific examples (a nonexhaustive list) of the computer-readable medium would include the following: an electrical connection (electronic) having one or more wires, a portable computer diskette (magnetic), a random access memory (RAM) (magnetic), a read-only memory (ROM) (magnetic), an erasable programmable read-only memory (EPROM or Flash memory) (magnetic), an optical fiber (optical), and a portable compact disk read-only memory (CD ROM) (optical). Note that the computer-readable medium could even be paper or another suitable medium upon which the program is printed, as the program can be electronically captured, via, for instance, optical scanning of the paper or other medium, then compiled, interpreted or otherwise processed in a suitable manner, if necessary, and then stored in a computer memory.

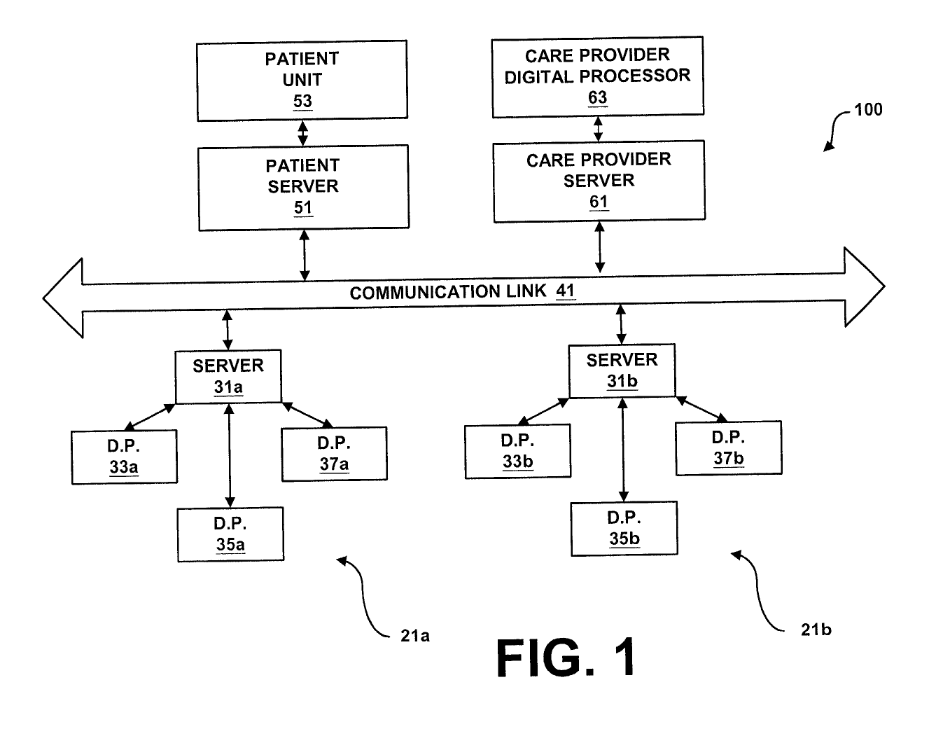

Preferably, the scoring and managing system of the present invention is implemented with use of the Internet. By way of example and illustration, FIG. 1 illustrates an Internet based system that may operate using a TCP/IP protocol, upon which the scoring and managing system 100 of the present invention may be implemented. It should be noted that while the present disclosure provides implementation of the scoring and managing system 100 within an Internet based system, the scoring and managing system 100 need not be provided via use of the Internet. Instead, one of reasonable skill in the art will appreciate that the scoring and managing system 100 may be implemented within other mediums, such as, for example, but not limited to, a local area network (LAN), or wide area network (WAN).

Further, in accordance with an alternative embodiment of the invention, the scoring and managing system 100 may instead utilize a multi-point control unit (MCU), wherein video conferencing systems located at several locations may be interconnected for conferencing between users, as described below. As known in the art, to initiate a conference using a MCU, a session host dials a number or makes some other appropriate connection such as a TCP/IP link, and then presents a conference identifier. The MCU then automatically sets up the conference and establishes TCP/IP connections to each user. Alternatively, users may then join the conference by dialing an access number to the MCU for instantaneous connection.

Referring to FIG. 1, a plurality of networks 21a, 21b are shown wherein each network 21a, 21b includes multiple digital processors 33, 35, 37. Digital processors 33, 35, 37 within each network 21a, 21b may include, but are not limited to, personal computers, mini computers, laptops, and the like. Each digital processor 33, 35, 37 is typically coupled to a host processor or server 31a, 31b for communication among processors 33, 35, 37 within the specific corresponding network 21a, 21b.

The host processor, or server, 31a, 31b is coupled to a communication link 41 that interconnects or links the networks 21a, 21b to each other, thereby forming an Internet. As such, each of the networks 21a, 21b are coupled along the communication link 41 to enable access from a digital processor 33a, 35a, 37a of one network 21a to a digital processor 33b, 35b, 37b of another network 21b.

Various end-user servers 51, 61, two of which are shown as an example, specifically, a patient server 51, and a care provider server 61, are linked to the communication link 41, thus providing the patient and care provider with access to the Internet. A care provider digital processor 63 is coupled to the care provider server 61 for purposes of allowing a care provider to interact with patients via the Internet, as is further explained hereinbelow. Likewise, a patient digital processor, or patient unit 53, is coupled to the patient server 51 for purposes of allowing a patient interact with the scoring and managing system 100 via the Internet, as is further explained hereinbelow.

Functionality of the scoring and managing system 100, in accordance with the preferred embodiment of the invention, is defined by a software program stored within the patient unit 53 and the care provider digital processor 63. The software program is operated on and connected to the Internet, for communication among the various networks 21a, 21b and/or digital processors 33, 35, 37 and other end-users connected to the Internet via respective end-user servers 51, 61. It should be noted that the patient unit 53 and care provider digital processor 63 may run any form of operating system, such as, but not limited to, Microsoft Windows, to support operation of the present scoring and managing system 100. Preferably, the networks 21a, 21b used by the scoring and managing system 100 are secure and encrypted for purposes of ensuring the confidentiality of information transmitted within and between the networks 21a, 21b.

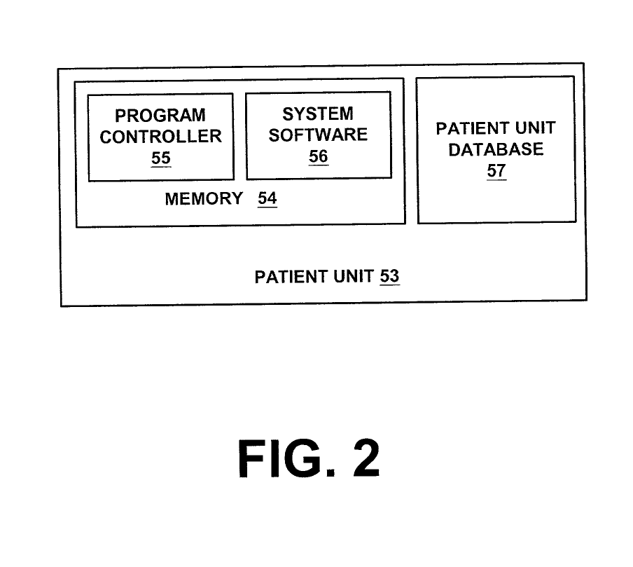

FIG. 2 is a block diagram that further illustrates the patient unit 53 of FIG. 1. As shown by FIG. 2, the patient unit 53 comprises a memory 54 having a program controller 55, and the scoring and managing system software 56. The program controller 55 is capable of performing functionality required by the scoring and managing system 100, as described in detail herein below. The patient unit 53 also comprises a patient unit database 57 that may store information such as, but not limited to, a name, address, medical conditions and treatment patterns of a patient. It should be noted that the patient unit database 57 may be located remote to the patient unit 53, wherein data may be stored by transmission to the remote location via the patient server 51. The locating and updating of data within the patient unit 53, as required by the scoring and managing system 100, is performed by the program controller 55.

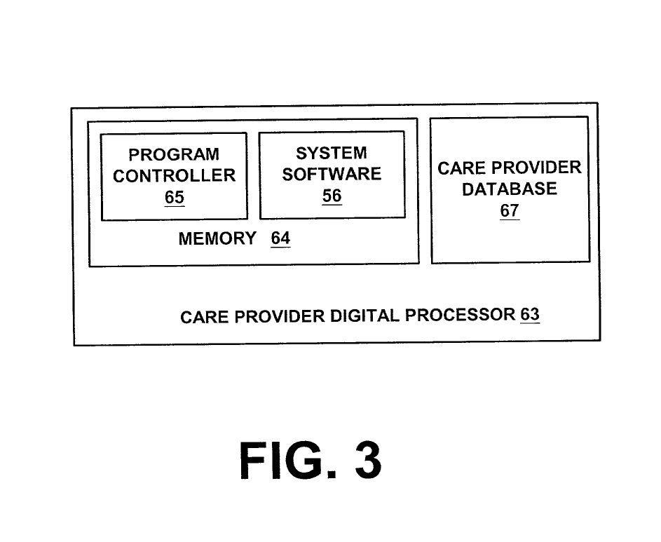

FIG. 3 is a block diagram that further illustrates the care provider digital processor 63 of FIG. 1. As shown by FIG. 3, the care provider digital processor 63 comprises a memory 64 having a program controller 65, and the scoring and managing system software 56. The program controller 65 is capable of performing functionality required by the scoring and managing system 100, as described in detail herein below. The care provider digital processor 63 also comprises a care provider database 67 for storing received patient information. It should be noted that the care provider database 67 may be located remote to the care provider digital processor 63, wherein data may be stored by transmission to the remote location via the care provider server 61. In fact, both the patient database 57 and the care provider database 67 may be located remote from the patient unit 53 and care provider digital processor 63, respectively, or even share a single database. The locating and updating of data within the care provider digital processor 63, as required by the scoring and managing system 100, is performed by the program controller 65.

The scoring and managing system software 56 defines logic for constructing and maintaining an individualized care plan by integrating a patient assessment process module, a disease management protocol module and a patient's medication compliance module. In essence, the software 56 defines an integrated set of assessment, data collection, and reporting tools. As described herein below, the care plan is designed to achieve specific patient goals that are directly related to disease process of the patient. Progress toward the established goals is monitored continuously by physicians and care providers who are capable of affecting modifications to the care plan of the patient as may be necessary to increase the ability of the patient to meet established outcome goals and avoid conditions that would result in unnecessary hospitalization.

The desired goals of a medical patient are not always clearly defined or target specific, instead goals are a set of long term and short term goals that are to be achieved by the patient during the course of care. These goals frequently need to be revised as the medical condition of the patient changes. The following provides a detailed description of the patient assessment process, disease management protocol and medical compliance of the patient, which are used in combination to obtain, and maintain, an individualized patient care plan in accordance with the preferred embodiment of the invention.

Patient Assessment Process Module

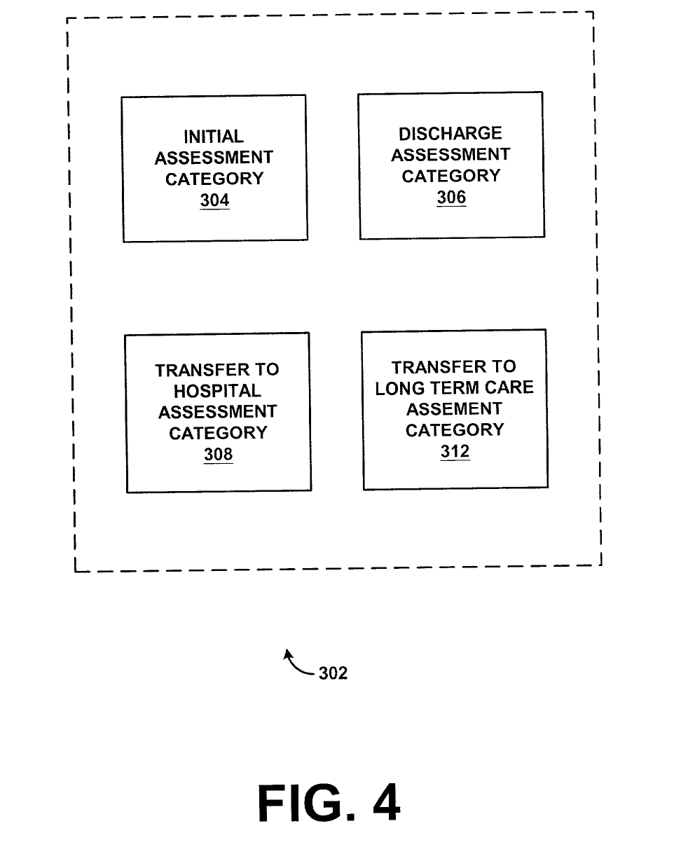

FIG. 4 is a block diagram that provides a logical depiction of the categories of data collected and maintained throughout a patient assessment process 302. It should be noted that other categories of information considered important in medically assessing a patient might be added or supplemented. It should also be noted that data entered, stored and updated, within the following categories may be stored in a table that is titled in accordance with the name of the category of data stored therein. As an example, a data table entitled discharge assessment may have data stored therein obtained during the discharge of a patient.

The patient assessment process 302 provides a standardized patient assessment based on protocol guidelines and standards of practice. A first category within the patient assessment process 302 is an initial assessment category 304. The initial assessment category 304 defines a series of assessment questions that are stored within the care provider database 67 (FIG. 3) and are answered by the care provider, a physician, or the patient. Preferably, these questions are answered at the time of initial patient assessment, which may be performed by a physician, nurse, or any other capable individual. The assessment questions are structured such that a specific series of assessment questions are associated with a specific patient disease. Identification of a patient disease lists a group of associated questions to be asked and answered accordingly. It should be noted that all assessments may be performed with the patient being physically located distant from the care provider, physician, or nurse.

A second category within the patient assessment process 302 is a discharge assessment category 306. The discharge assessment category 306 defines a series of assessment questions that are stored within the care provider database 67 and are to be answered by the care provider, physician, or the patient at the time of patient discharge. Preferably the discharge assessment questions contain many of the same questions as those asked within the initial assessment category 304, thereby providing a means of comparison for assessment of patient medical performance. Like the questions asked within the initial assessment category 304, questions asked within the discharge assessment category 306 are structured such that a specific series of questions are associated with a specific patient disease. Identification of the patient disease preferably lists a group of associated questions to be asked and answered accordingly.

A third category within the patient assessment process 302 is a transfer to hospital assessment category 308. The transfer to hospital assessment category 308 defines a series of assessment questions that are stored within the care provider database 67, and are to be answered by either the care provider, physician, or the patient at the time of a patient being transferred to a hospital. The questions associated with the transfer to hospital assessment category 308 are associated with a specific patient disease which are to be answered at the time of the patient transferring to a hospital, or, alternatively, transferring between hospitals. Preferably, identification of the patient disease lists a group of associated questions to be asked and answered accordingly.

A fourth category within the patient assessment process 302 is a transfer to long-term care assessment category 312. The transfer to long-term care assessment category 312 defines a series of assessment questions that are stored and are to be asked and answered at the time of a patient being transferred to long term care. An example of long-term care may include, but is not limited to, treatment provided to a patient having a terminal illness, which requires long term treatment. The questions associated with the transfer to long-term care assessment category 312 are structured such that a specific series of questions are associated with a specific patient disease. Preferably, identification of a patient disease lists a group of associated questions to be asked and answered accordingly. It should be noted that other customized categories of assessments might be used in association with the abovementioned categories.

Analysis of the above-mentioned assessment questions and answers within the patient assessment process 302 provides a detailed vehicle for determining patient progression throughout medical treatment. As an example, a physician or care provider may compare answers provided during initial assessment, to answers received during discharge. A thorough analysis of the answers provided to the assessment questions allows a physician or care provider to determine whether a patient is recovering properly according to the care plan of the patient, as described in detail hereinbelow, and if needed, to change the care plan of the patient accordingly.

Disease Management Protocol Module

The disease management protocol module, as a second portion of the patient care plan, is an automated database of accepted medical guidelines for the treatment and care of specified disease conditions. These guidelines indicate a course of treatment and care that is recommended for a disease condition in order to achieve a positive patient outcome. These guidelines are preferably provided in an automated format such that the disease management protocol module may be used by the scoring and managing system 100.

The disease management protocol module provides a course of treatment and care that suggests specific medical management devices to be used, and the frequency of such use. The use of the medical devices may then be modified based upon the previously performed patient assessment 302, abilities of the patient, and the prior medical experience of the care provider. Preferably, modification of medical device usage is performed by a care provider working in coordination with a physician. The course of treatment and care also suggests a nominal schedule enumerating a specific number of visits necessary to complete the prescribed treatment for the patient within the specified care plan. Since the nature of the scoring and managing system 100 (i.e., use over the Internet) allows a care provider to “visit” with a patient from a location distant from the patient, a greater number of “visits” can be made over a shorter period of time.

The course of treatment and care also specifies activities to be accomplished and assessed during “visits” with the patient. The medical measurement devices, schedule, and activities are all capable of being modified for use in the care plan for a particular patient based on information gathered about the patient during patient assessment and medication compliance assessment (shown hereinbelow). Therefore, the care plan remains current to the specific medical conditions of a specific patient while enhancing recovery.

Medication Compliance Module

The medication compliance model, as a third portion of the patient care plan, operates in a real-time or intermittent access networked environment where the patient, physician, and care provider have access, local or distant, to a common data storage area. The medication compliance module provides access to, and a listing of, current medications being taken by the patient, the medical history of the patient, and review documents of medication administration. Further, the medication compliance module determines the level of medication supervision required for effective patient compliance, and monitors the medical inventory of the patient such that a reorder process may be performed upon medication depletion.

The principal intent of the patient medication compliance module is to monitor and influence patient medication compliance for beneficial results. An example of a possible implementation of a process used for managing the use of prescribed medication by a patient is described in co-pending commonly assigned application entitled “A System and Method for Providing Medication Management,” by John Edward Haines, Walter Charles Hayhurst, Jr. and Linda L. Roman filed on [UNFILED], and having attorney docket number 050320-1050, the disclosure of which is incorporated herein by reference.

Medication compliance by the patient is assessed and monitored via use of the medication compliance module in a near-real-time environment, namely, the Internet. The level of intervention is determined in a standardized manner, and allows the intensity of the intervention with the patient to be tailored to the particular needs of the patient.

Preferably a scoring method is used for qualifying patients for enrollment into the scoring and managing system 100. The scoring method comprises assessment questions that are customized to assess a need for a patient to use of the present system 100. The assessment questions contain associated scores that may be accumulated upon completion of the assessment questions, resulting in a total score which is representative of a level of care provider interaction necessary to assist the patient in achieving specific medical goals.

A patient's care plan may be adjusted in accordance with the score received after completion of the assessment questions. Therefore, as disclosed in the application entitled “A System and Method for Providing Medication Management,” incorporated herein by reference, if a score obtained by a patient is, for example, low, the low score may correspond to a need for constant supervision of the patient during medication intake or application. A care provider may provide this supervision. As a result, the patient may be required to have daily video conferences with the care provider. It should be noted that the scoring method may associate a point value to questions asked within the patient assessment protocol module, in addition to assessment questions asked to determine a need of a patient to be registered with the present system 100. Thereafter, the separate scores may be compared to determine progress in addressing the medical condition of the patient, and appropriate care plan modifications made thereafter. In this manner, the scoring method allows the patient assessment protocol module to be used as a monitoring mechanism.

The accumulated data from qualifying assessment questions, including received scores, combined with the initial and continuing patient assessment processes, allows for objective evaluation of the progress of the patient. The effectiveness of the care plan can then be assessed in order to improve the applied modules accordingly and the care plan adjusted to maximize medical recovery. In other words, the ability of the scoring and managing system 100 to continuously and accurately collect data regarding the patient and activities of the patient provides a critical advantage in the management of patient recovery.

The level of intervention recommended by the medication compliance module influences the activities conducted as part of the care plan. A high level of non-compliance connotes a requirement for more intense interaction with the patient to encourage compliance and avoid negative medical results. It also provides an objective set of criteria for determining progress toward the patient goals and the desired outcome.

The scoring and managing system 100 allows for near real time adjustments to be applied to the treatment and care of a patient, as needed. When it is noted that the condition of a patient deviates from the trend or path necessary to achieve specific medical goals, the care provider can immediately adjust the care plan to meet the medical needs of the patient. If the patient is showing better progress than anticipated, the schedule and intensity of interventions can be reduced. However, as mentioned hereinabove, if the patient is not progressing as anticipated or begins to develop indications of a secondary condition, the physician may be called upon to modify the care plan of the patient as required.

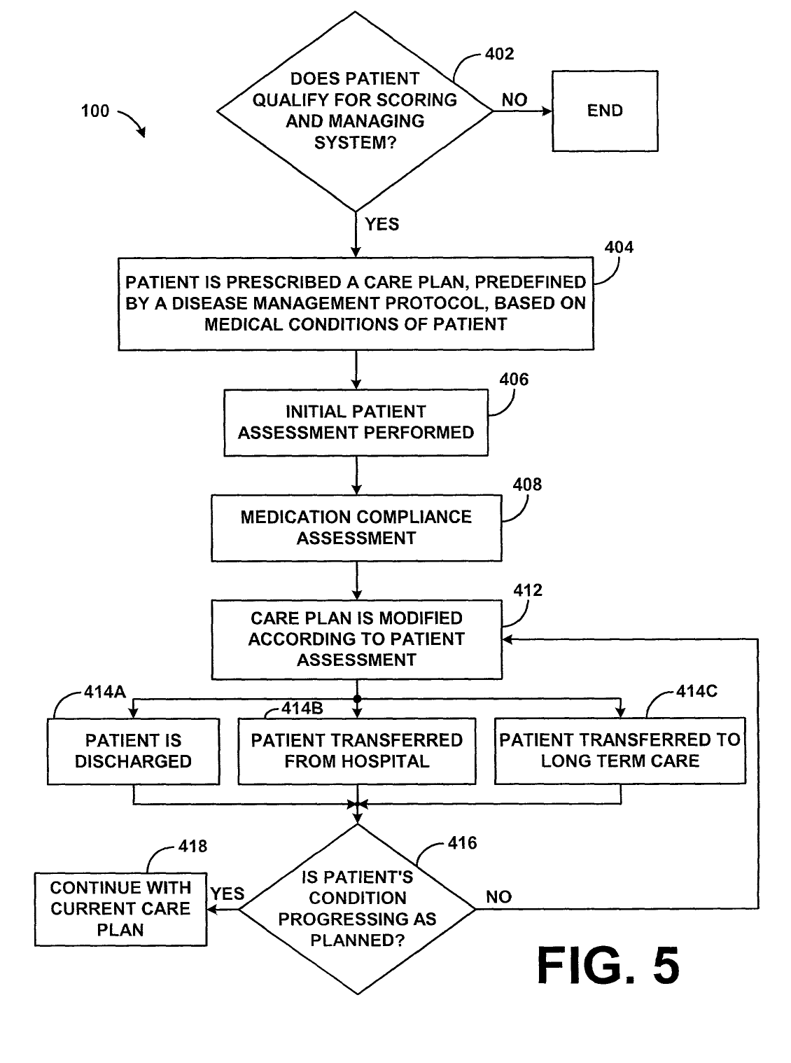

FIG. 5 is a flow chart that illustrates a logical path taken by the scoring and managing system 100 to ensure rapid patient recovery. With regard to the flow chart of FIG. 5, each block represents a module, segment, or portion of code, which comprises one or more executable instructions for implementing the specified logical function(s). It should also be noted that in some alternate implementations, the function(s) noted in the blocks might occur out of the order noted. For example, two blocks shown in succession may in fact be executed substantially concurrently, or the blocks may sometimes be executed in the reverse order, depending upon the functionality involved.

Referring to FIG. 5, a determination is first made as to whether a patient qualifies for partaking in the scoring and management system 100 (block 402). An initial consultation, or a series of consultations, takes place between a caregiver and a patient having a medical condition requiring treatment or attention. Based upon the characteristics of the medical condition of the patient during the initial consultation, use of the present scoring and management system 100 may be required as part of the treatment for the medical condition. Preferably, the present remedial system 100 is made available to patients that require monitoring as part of treatment for their medical condition.

As shown by block 404, if the patient qualifies for partaking in the scoring and managing system 100, the patient is prescribed a care-plan based upon the medical conditions of the patient. Selection of an appropriate initial care-plan is performed by the disease management protocol that selects an appropriate care-plan from a predefined listing of care-plans, wherein at least one care-plan is associated with the treatment of a specific disease.

As shown by block 406, an initial patient assessment is then performed, as described hereinabove, wherein a series of questions are asked and answered so as to determine all current aspects of the medical condition of the patient prior to beginning participation in the prescribed care-plan. As mentioned hereinabove, the answers to each question are assigned a score for future comparison purposes. A medication compliance assessment of the patient is then performed to determine a level of medication supervision required for effective medication compliance by the patient (block 408).

Based upon the results of the initial patient assessment (block 406) and medication compliance assessment (block 408), the care-plan may be modified such that the resulting patient care-plan is personalized to the assessed patient (block 412). Since, in accordance with the preferred embodiment of the invention, the initial patient assessment (block 406) and medication compliance assessment (block 408) may be performed via the Internet via use of the patient unit 53 and care provider digital processor 63, the care plan of the patient may be modified almost instantaneously. The versatility of the care plan of the patient provides the patient with a vehicle for ensuring that the patient receives the best medical care possible throughout treatment of the medical condition of the patient.

As shown by blocks 414A, 414B, and 414C, the scoring and managing system 100 performs a series of queries, associated with scores, at predetermined instances to determine whether the medical status of a patient has changed since initial assessment (block 406). As an example, block 414A shows that a determination of whether the patient has been discharged from the place of initial assessment, such as a hospital, is made. If the patient is being discharged, a new patient assessment is performed. Similarly, block 414B shows that a determination of whether the patient has been transferred from one hospital to another is made. If the patient is being transferred from one hospital to another, a new patient assessment is performed. Still further, block 414C shows that if a patient is transferred to a long-term care facility a new patient assessment is performed. It should be noted that the scoring and managing system 100 may be modified such that other questions may be asked that are representative of periods in time that are significant in treatment of a patient. Alternatively, a new patient assessment may be performed in accordance with the scoring and managing system 100 after a specified period of time to determine the progress of a patient as a factor of time and treatment.

As shown by block 416, a determination is then made as to whether the medical condition of the patient has been progressing as planned. As shown by block 418, if the medical condition of the patient has been progressing as planned, treatment of the patient continues in accordance with the current care-plan. However, if the condition of the patient has not progressed as planned the care-plan of the patient is modified according to the newest patient assessment (block 412). Therefore, the patient's care-plan changes in accordance with the most recent patient assessment performed. Further, since these changes take place via the Internet, there is no reason for the patient to be burdened by continuous hospital visits for re-assessment.

It should be emphasized that the above-described embodiments of the present invention, particularly, any “preferred” embodiments, are merely possible examples of implementations, merely set forth for a clear understanding of the principles of the invention. Many variations and modifications may be made to the above-described embodiment(s) of the invention without departing substantially from the spirit and principles of the invention. All such modifications and variations are intended to be included herein within the scope of this disclosure and the present invention and protected by the following claims.

What is claimed is:

1. A system for scoring and managing patient progression, comprising:
a transceiver;
a memory communicably coupled to said transceiver, having logic defining functions to be performed by said system;
a processor configured by said memory to perform the functions of:
prescribing a care plan for said patient that is predefined and based upon medical conditions of said patient;
performing an initial medical assessment of said patient;
performing a medication compliance assessment of said patient;
modifying said care plan based upon said initial assessment and said medication compliance assessment; and
modifying said care plan in accordance with new assessments performed in association with medical progress of said patient.

2. The system of claim 1, wherein said initial medical assessment is performed via use of information received by said transceiver.

3. The system of claim 1, wherein said transceiver allows a care provider to assist in managing said patient progression.

4. The system of claim 1, wherein said new assessments are performed at a time selected from the group consisting of: patient discharge; patient transferal from a hospital; and patient transferal to long term care.

5. The system of claim 1, wherein said transceiver communicates via the Internet.

6. The system of claim 1, wherein said memory and processor are located within a computer that is located within a local area network.

7. The system of claim 1, wherein said initial medical assessment comprises a series of questions asked of said patient for purposes of determining medical conditions and identity of said patient, and wherein answers to said questions are awarded a score for purposes of future comparison with answers received during said new assessments.

8. The system of claim 7, wherein said initial medical assessment is provided via the Internet.

9. The system of claim 1, wherein said care plan is selected from a database of pre-defined care plans that are identified by medical conditions, said care plan providing medical guidelines for the treatment and care of said medical conditions.

10. The system of claim 1, wherein said medication compliance assessment determines a level of intervention necessary to assure that said patient conforms with medication usage prescribed by said care plan.

11. A method for scoring and managing patient progression, comprising the steps of:
determining whether said patient qualifies for participating in a program using said method;
prescribing a care plan for said patient that is based upon medical conditions of said patient;
performing an initial medical assessment of said patient;
performing a medication compliance assessment of said patient;
modifying said care plan based upon said initial assessment and said medication compliance assessment; and
modifying said care plan in accordance with new assessments performed in association with medical progress of said patient.

12. The method of claim 11, wherein said new assessments are performed at a time selected from the group consisting of: patient discharge; patient transferal from a hospital; and patient transferal to long term care.

13. The method of claim 1, wherein said step of performing an initial medical assessment comprises a series of questions asked of said patient for purposes of determining medical conditions and identity of said patient, and wherein answers to said questions are awarded a score for purposes of future comparison with answers received during said new assessments.

14. The method of claim 13, wherein said step of providing an initial medical assessment is provided via the Internet.

15. The method of claim 11, wherein said step of determining whether said patient qualifies for participating in said program is performed by providing an initial consultation with said patient via the Internet.

16. The method of claim 11, wherein said care plan is selected from a database of pre-defined care plans that are identified by medical conditions, said care plan providing medical guidelines for the treatment and care of said medical conditions.

17. The method of claim 11, wherein said step of performing an initial medical assessment is performed by said patient physically visiting a care provider.

18. A system for scoring and managing patient progression, comprising:
means for prescribing a care plan for said patient based upon medical conditions of said patient;
means for performing an initial medical assessment of said patient;
means for performing a medication compliance assessment of said patient;
means for modifying said care plan based upon said initial assessment and said medication compliance assessment; and
means for modifying said care plan in accordance with new assessments performed in association with medical progress of said patient.

19. The system of claim 18, wherein said care plan is predefined.

20. The system of claim 18, wherein said means for performing an initial medical assessment uses the Internet to perform said assessment.

21. The system of claim 18, wherein said new assessments are performed at a time selected from the group consisting of: patient discharge; patient transferal from a hospital; and patient transferal to long term care.

22. The system of claim 18, wherein said means for performing an initial medical assessment utilizes a series of questions asked of said patient for purposes of determining medical conditions and identity of said patient, and wherein answers to said questions are awarded a score for purposes of future comparison with answers received during said new assessments.

23. The system of claim 18, wherein said means for determining whether said patient qualifies for participating in said program uses the Internet to provide an initial consultation with said patient for said determination.

24. The system of claim 18, wherein said care plan is selected from a database of pre-defined care plans that are identified by medical conditions, said care plan providing medical guidelines for the treatment and care of said medical conditions
